(19) United States
(12) Patent Application Publication
(10) Pub. No.: US 2007/0238934 A1
(43) Pub. Date: Oct. 11, 2007

(54) Dynamically responsive mood sensing environments

(76) Inventor: Tarun Viswanathan, Bangalore (IN)

Correspondence Address: SCHWABE, WILLIAMSON & WYATT, P.C., PACWEST CENTER, SUITE 1900, 1211 S.W. FIFTH AVE., PORTLAND, OR, 97204, US

(21) Appl. No.: 11/394,599

(22) Filed: Mar. 31, 2006

(51) Int. Cl. A61B 5/00

(52) U.S. Cl. 600/300

(57) ABSTRACT

Physiological readings, such as heart rate, breathing rate, pulse, blood pressure, electrical conductivity across skin surface(s), electrical or magnetic activity of various regions of the brain, body temperature, voice patterns, voice stress, blood chemistry, locomotion (walking, running, etc.) characteristics, eating patterns, personal movement, etc. may be monitored for one or more users. It will be appreciated locomotion may be determined, for example, by way of sensors in shoes or walking surfaces, as well as by motion picture modeling analyzing one's motion as seen by one or more cameras placed in the environment. It will be appreciated these exemplary attributes may be used to infer or estimate one's current mood or interests directly or based on models. An adaptive environment may be used to refine models to improve mood determination accuracy, and to configure devices in the environment according to a determined mood.

### FIELD OF THE INVENTION

The invention generally relates to monitoring physiology to estimate one's mood, and more particularly to configuring devices, such as consumer electronics, based at least in part with respect to a determined mood.

### BACKGROUND

Recently, more research and development attention has been paid to integrating devices, such as consumer electronic devices, in to “digital homes” or “digital offices” and other environments (hereafter generally “digital environment”) in which synergistic results are desired through use of resources offered by various devices in an environment. In one environment, devices are designed to respond to user interests, such as to provide entertainment in accord with a user's designated interests. One well known example of this is Bill Gates' Washington state home; apparently Mr. Gates may use a pre-coded “pin” or marker to signify his current interest, and as he walks through his home, various devices within the home respond to the interest designated by his pin; for example, apparently the home is equipped with digital screens present different types of artwork in accord with his designated interests, and music that is played in accord with his designated interest.

Unfortunately, requiring selection of a pin or other device to indicate to a digital environment one's current interest is limiting, especially if one's interest changes depending on what one is doing. This may be alleviated, as will be discussed in the detailed description below, by extending a technology used for medical health monitoring.

### BRIEF DESCRIPTION OF THE DRAWINGS

The features and advantages of the present invention will become apparent from the following detailed description of the present invention in which:

FIG. 1 illustrates a system according to one embodiment including sensors for tracking state data and a device manager for configuring devices in accord with a perceived mood determined at least in part on the tracked state data.

FIG. 2. illustrates a flowchart according to one embodiment for entering an environment and having devices adjusted to suit one's estimated mood.

FIG. 3 illustrates a suitable computing environment in which certain aspects of the invention may be implemented.

### DETAILED DESCRIPTION

The Fraunhofer Institute for Open Communikation Systems of Berlin, Germany, for example, provides devices to monitor vital signs such as heart rate, where distress status may be inferred from the monitoring and appropriate responsive action taken as needed, e.g. calling paramedics. To perform monitoring, technology is used to provide what is sometimes referred to as a Body Area Network (BAN), where sensors are applied to one's body and readings provided to one or more associated devices to track one's physiological status.

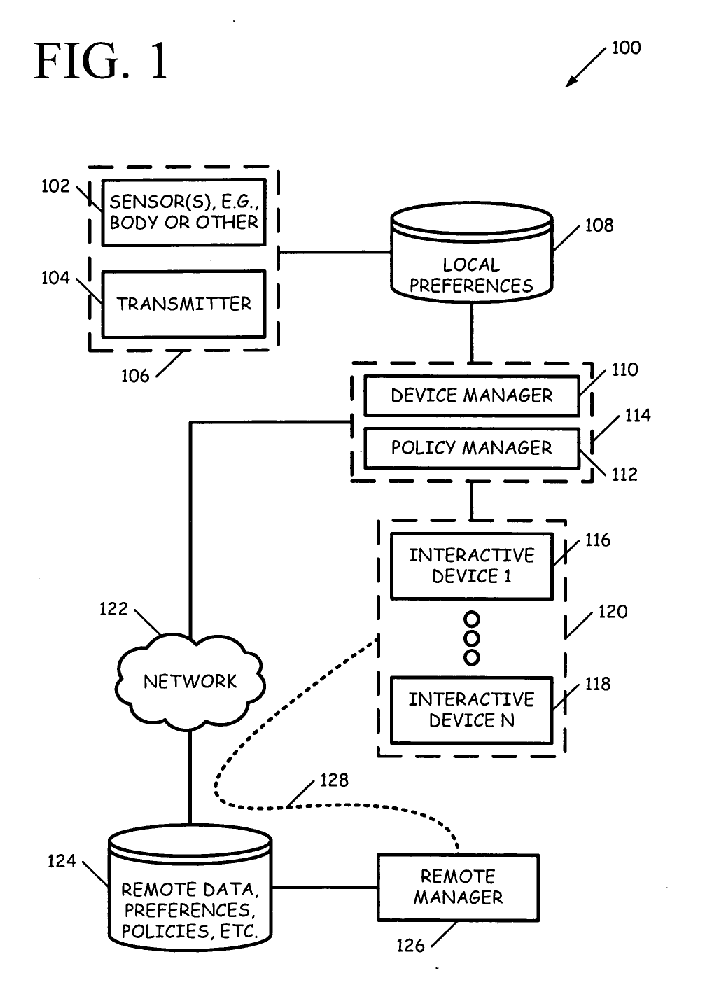

In various embodiments discussed below, the BAN concept and other such techniques are extended to include monitoring many physiological readings, including heart rate, breathing rate, pulse, blood pressure, electrical conductivity across skin surface(s), electrical or magnetic activity of various regions of the brain, body temperature, voice patterns, voice stress, blood chemistry, locomotion (walking, running, etc.) characteristics, etc. It will be appreciated locomotion may be determined, for example, by way of sensors in shoes or walking surfaces, as well as by motion picture modeling analyzing one's motion as seen by one or more cameras placed in the environment. It will be appreciated that these are just an exemplary few of the attributes of physiological state that may be monitored and others are noted in FIG. 1 as well.

In various embodiments, these physiological readings may be used to infer or estimate one's current mood or interests. In some embodiments, physiological readings may be interpreted with respect to one or more models from which one's current mood and/or interests may be determined. For example, it will be appreciated by one skilled in the art that experts studying the interplay between psyche and physiology, such as psychologists, psychiatrists, etc., have for years been developing models and profiles to correlate moods, interests, attitudes and/or desires with physiological state.

An adaptive environment may be used to refine models to improve mood determination accuracy. For example, an adaptive environment may monitor various physiological aspects of a user and periodically interacting with the user to determine the user's mood. For example, a user may wear a transceiver and when a physiological change is noted, such as may occur responsive to sudden elation, anger, sadness, etc., the environment may query the user, e.g., by audible questions and voice recognition responses, through an interface associated and/or on the transceiver, etc., to ask the user what the physiological change represents.

Further, with use of smart consumer devices such as refrigerators or freezers that track their contents, the presence or introduction of “comfort foods” may be used in part to infer one's current mood or well being. Similarly, the nature of interaction with various devices such as televisions and telephones may be used to infer mood, including tactile feedback data for a device such as squeezing it hard, banging its buttons, throwing it, etc. Also, for entertainment devices such as televisions, radios, etc., a genre of music selected on music channels, or television show selected may be used at least in part to infer mood. Also, past experience with certain telephone numbers called or calls received from may correlate with responsive mood changes. All of these techniques and physiology monitoring may be used in various embodiments to refine and define mood determination models.

FIG. 1 illustrates a system 100 according to one embodiment. Illustrated are sensors 102, which as discussed may be body sensors tracking physiological state, or other data, such as gait information, etc. Associated with the sensor 102 is one or more transmitters 104 (only one is illustrated) for conveying data identified by the sensor. It is assumed sensors may utilize wireless communication for convenience, however they may be wired. The dashed region 106 is to illustrate that sensors and transmitters may be disposed within a single device or housing, or they may be separate devices associated with each other.

A data store 108, such as a database, may be used to store preference data, policies, etc. that may be used in determining how to manage or configure one or more devices based on sensor 102 data. Preferences for individuals' moods may include, for example, playing specific songs or music genre when a person is believed to be stressed, playing or suggesting playing specific movies or genre of movie when a person is believed, for example, to be in a good mood, setting light settings for various determined moods, setting up a hot water bath (assuming electronic control of fixtures) or recommending a hot bath by way of a user interface for certain moods or conditions, such as when tired, etc.

As discussed above, sensor and other determined data may be used to estimate a mood for a person associated with one or more sensors. Preference data and/or policies may be present in the data store 108 that indicate how to configure certain devices in accord with an estimated mood. It will be appreciated that the data store may be a stand alone device, such as network attached storage (NAS), or storage within another device. While one can always manually select a mood, as discussed above, mood may be estimated based on various factors such as heart rate, adrenaline level in one's body, blood pressure, breathing rate, one's movement characteristics (e.g., gait), how one interacts with devices (e.g., throwing, squeezing hard, banging), voice levels (e.g., stress, volume, speech speed), one's skin electrical conductivity, perspiration rate, facial expression, electromyographic (EMG) measurement, etc.

For example, although more complex models may be employed, an individual's heart rate is typically slowed when relaxed or in a good mood, hence a change going above a typical baseline rate may be a factor in concluding one has become agitated, perhaps angry, or if below typical then it may be a factor in concluding one has become depressed or sad. Breathing rate may also vary and become a factor in estimating mood. For example, if one's breathing rises above a typical baseline rate, then it may be a factor in concluding one has become agitated, angry, etc., and if below typical then it may be a factor in determining depression, frustration, ennui, etc. Measured brain activity may also be used as a factor in determining mood. For example, electroencephalograph (EEG) or magneto encephalography (MEG) data may be captured through use of sensors attached to or proximate to one's scalp and amplified voltage or magnetic variations measured and recorded. Rhythms or levels of electrical activity may be a factor in estimating one's mood. For example, when a person is in an unpleasant mood, there can be a measurable change in EEG activity of one's brain over a typical baseline measurement. Baseline values for various measurable physiological data may be recorded in the data store 108, as well a recording history to allow analyzing the data for trends or other data characteristics in addition to using the data as needed to perform mood determination.

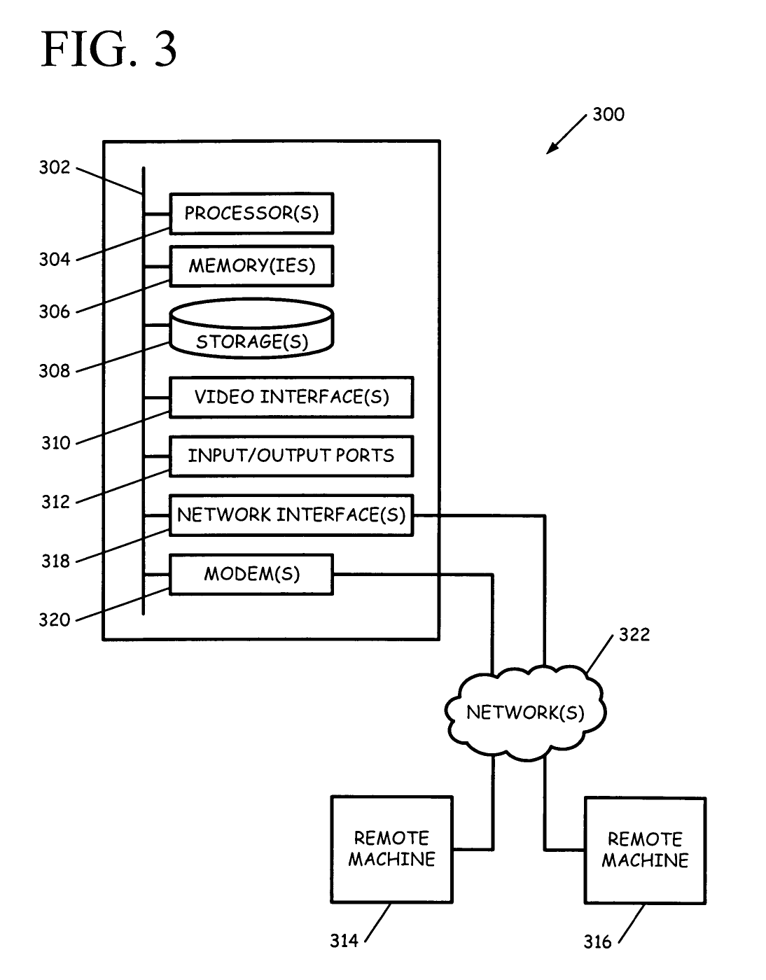

A device manager 110 and/or a policy manager 112 may be communicatively coupled with one or more data stores 108 (only one is illustrated) and one or more sensors 102. In the illustrated embodiment, the device manager 110 is responsible for programming and/or configuring devices 116, 118 as a person's mood changes. It will be appreciated that device configuration may be location specific, e.g., devices may be configured as a user enters a region, such as a room, and if desired, reconfigured when the user leaves the region. The device manager may be communicatively coupled with configurable devices 116, 118 by way of a local area network (LAN), including wired and wireless communication environments, or a variety of other techniques, see e.g. FIG. 3 network 322.

The policy manager, which may be incorporated into a single device 114 with the device manager, can be used to access policies to direct application of preferences that may be applied by the device manager 110. For example, even though a preference may indicate a certain kind of music genre for a particular mood, a policy may direct that only certain subsets of the music be chosen at certain times of the day. For example, upbeat or high-tempo music might be restricted by policy to only be played during the day, while more sedate music is required to be chosen at night. Policies may also incorporate, of course, access controls to determine who may perform what device configurations, and in what environments. Thus, for example, parents could limit child access to device reconfiguration to particular devices and/or locations. Not illustrated but possibly inherent to the device manager 110 and/or policy manager 112 is an expert system for receiving sensor 102 input from transmitter(s) 104 and to control configuring devices 120 in accord with the policy manager 112 and applicable policies.

It will be appreciated that while devices 102, 104, 108, 112, 120 (hereafter “local devices”) may operate in a self contained environment, such as a digital home, digital office, or the like, these local devices may also be communicatively coupled by way of a network 122 such as the Internet, to remote devices that may serve as a remote control for the local devices, such as by a remote manager 126 operating as indicated by the dashed line to direct 128 operation of selected devices 120 through the network 122. The remote manager may also serve as input to the operations of the device manager 110 and/or policy manager 112. Thus, for example, the illustrated system provides for roaming users, where one may leave one's preferences, policies, etc. in a remote data store 124 that may be read by the device manger 110 when one enters the domain of the local devices.

Or, for example, if you travel from your house to a friend's house, your preferences may be retrieved when you enter the friend's house to control devices 120. However, as noted above, policy manager 112 may have a policy or policies restricting or preventing such roaming. In similar fashion, public access locations, such as lounges, wired-for-sound seating, portable electronic devices, media devices, games, etc. may all be configured to operate in accord with your interests as indicated by data in remote store 124. For example, a rental market may be provided allowing you to rent a media entertainment device that immediately operates in accord with your preferences, interests and/or mood assuming the rented device or current environment contains sensor(s) 102.

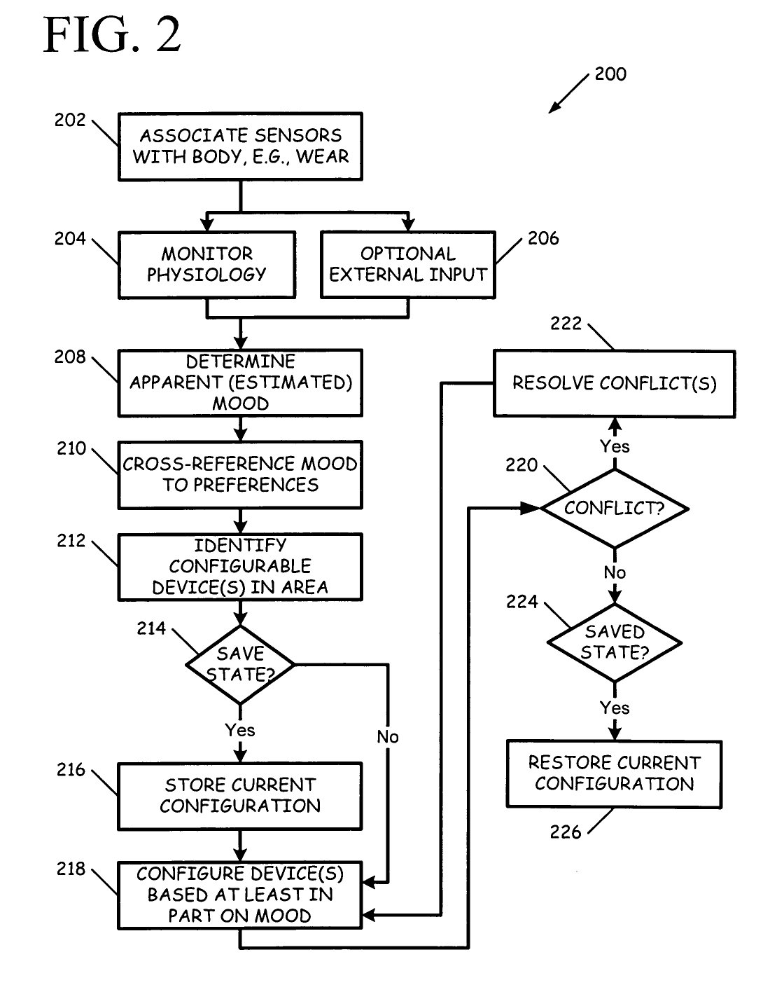

FIG. 2. illustrates a flowchart 200 according to one embodiment for entering an environment and having devices, e.g., FIG. 1 items 102, adjusted to suit one's mood. As illustrated, sensors such as FIG. 1 item 102 are associated 202 with a body of a user to monitor 204 the user's physiology. Sensors external to a user may also form input 206 to assist with analyzing a user's mood or other state information.

For example, sensors can also be worn by a user, or be proximate to the user, such as in wireless sensors affixed to shoes or other tools. Sensors may also be installed in the environment and used in part to analyze the user. For example, video cameras may be used from which video data can be analyzed for characteristics of a user's movement, for performing facial recognition and/or facial tomography analysis, microphones maybe used to capture voice exemplars for voice analysis, thermal sensors to monitor the user's heat output, motion detectors or radio frequency identifier (RFID) or similar technology may be used to track the user's movement in an environment such as a home, office, warehouse, or any other location.

It will be appreciated that a marker or other device may be used to easily distinguish between different users of disclosed embodiments of the invention. However, it will be appreciated by one skilled in the art that a combination of physiological features, including analysis of one's facial and/or body physiology may be used to generate a unique identifier for a user so that the user's data may be automatically retrieved, such as from FIG. 1 data stores 108, 124, when the user enters an environment and without the user having to have a particular marker or other device identifying the user. Thus, multiple members of a family residence, a workplace, or other environment, may transparently utilize the disclosed system and techniques.

Based at least in part on monitored 204 physiology and/or external input 206, a user's mood may be determined 208. As discussed above, various techniques and models may be used to determine an apparent mood (or other user state of interest) from monitored 204, 206 data. In the illustrated embodiment, an apparent or estimated mood is cross-referenced 210 to preferences. As noted above, the preferences may be accessed to determine how to configure devices responsive to a particular mood or other user state. After identifying 212 configurable devices in the user's area, such as digital picture frames, or music devices, etc., a test may be performed regarding whether 214 to save 214 a current state for the devices in the user's area. Thus, for example, devices may be performing a task, and if 214 state is to be saved, the state can be stored 216 before the devices are repurposed when they are configured 218 based at least in part on the user's estimated 208 mood.

It will be appreciated that configuring 218 a device may result in a conflict, such as when devices to be configured or an environment has local policies that conflict with the user's preferences, or when desired songs, genres, volumes, etc. are unavailable with devices available to the user for configuration. If 220 there is some conflict, then when possible, conflicts are resolved 222 and processing continues with configuring 218 devices. After configuring devices, if 224 there was a saved state, the stored device configuration(s) are restored 226.

FIG. 3 and the following discussion are intended to provide a brief, general description of a suitable environment in which certain aspects of the illustrated invention may be implemented. As used herein below, the term “machine” is intended to broadly encompass a single machine, or a system of communicatively coupled machines or devices operating together. Exemplary machines include computing devices such as personal computers, workstations, servers, portable computers, handheld devices, e.g., Personal Digital Assistant (PDA), telephone, tablets, etc., as well as transportation devices, such as private or public transportation, e.g., automobiles, trains, cabs, etc.

Typically, the environment includes a machine 300 that includes a system bus 302 to which is attached processors 304, a memory 306, e.g., random access memory (RAM), read-only memory (ROM), or other state preserving medium, storage devices 308, a video interface 310, and input/output interface ports 312. The machine may be controlled, at least in part, by input from conventional input devices, such as keyboards, mice, etc., as well as by directives received from another machine, interaction with a virtual reality (VR) environment, biometric feedback, or other input source or signal.

The machine may include embedded controllers, such as programmable or non-programmable logic devices or arrays, Application Specific Integrated Circuits, embedded computers, smart cards, and the like. The machine may utilize one or more connections to one or more remote machines 314, 316, such as through a network interface 318, modem 320, or other communicative coupling. Machines may be interconnected by way of a physical and/or logical network 322, such as an intranet, the Internet, local area networks, and wide area networks. One skilled in the art will appreciated that communication with network 322 may utilize various wired and/or wireless short range or long range carriers and protocols, including radio frequency (RF), satellite, microwave, Institute of Electrical and Electronics Engineers (IEEE) 802.11, Bluetooth, optical, infrared, cable, laser, etc.

The invention may be described by reference to or in conjunction with associated data such as functions, procedures, data structures, application programs, etc. which when accessed by a machine results in the machine performing tasks or defining abstract data types or low-level hardware contexts. Associated data may be stored in, for example, volatile and/or non-volatile memory 306, or in storage devices 308 and/or associated storage media, including conventional hard-drives, floppy-disks, optical storage, tapes, flash memory, memory sticks, digital video disks, etc., as well as more exotic mediums such as machine-accessible biological state preserving storage. Associated data may be delivered over transmission environments, including network 322, in the form of packets, serial data, parallel data, propagated signals, etc., and may be used in a compressed or encrypted format. The phrase “machine readable medium” shall accordingly include, but not be limited to, solid-state memories, optical and magnetic disks, and a carrier wave that encodes a data signal. Associated data may be used in a distributed environment, and stored locally and/or remotely for access by single or multi-processor machines. Associated data may be used by or in conjunction with embedded controllers; hence in the claims that follow, the term “logic,” if recited, is intended to refer generally to possible combinations of associated data and/or embedded controllers.

Thus, for example, with respect to the illustrated embodiments, assuming machine 300 embodies the Device Manager 110 of FIG. 1, then remote machines 314, 316 may respectively be one or more of the FIG. 1 configurable devices 120, or the Remote Manager 126. It will be appreciated that remote machines 314, 316 may be configured like machine 300, and therefore include many or all of the elements discussed for machine.

Having described and illustrated the principles of the invention with reference to illustrated embodiments, it will be recognized that the illustrated embodiments can be modified in arrangement and detail without departing from such principles. And, though the foregoing discussion has focused on particular embodiments, other configurations are contemplated. In particular, even though expressions such as “in one embodiment,” “in another embodiment,” or the like are used herein, these phrases are meant to generally reference embodiment possibilities, and are not intended to limit the invention to particular embodiment configurations. As used herein, these terms may reference the same or different embodiments that are combinable into other embodiments.

Consequently, in view of the wide variety of permutations to the embodiments described herein, this detailed description is intended to be illustrative only, and should not be taken as limiting the scope of the invention. What is claimed as the invention, therefore, is all such modifications as may come within the scope and spirit of the following claims and equivalents thereto.

What is claimed is:

1. A method for a mood sensing environment containing adjustable machines to dynamically respond to mood changes of a user, comprising:
associating one or more sensors with a user, at least one of said sensors monitoring one or more physiological conditions of the user;
determining a mood for the user based at least in part on monitored physiological conditions of the user;
selecting a configurable machine of the environment; and
changing a configuration of the configurable machine based at least in part on the determined mood of the user.

2. The method of claim 1, further comprising automatically changing the configuration responsive to changes in the user's mood.

3. The method of claim 1, further comprising:
associating a body area network with the user, the body area network including selected ones of said one or more sensors associated with the user.

4. The method of claim 1, further comprising:
determining a policy applicable to the mood sensing environment; and
changing the configuration in accord with the policy.

5. The method of claim 4, wherein the policy includes configuration preferences associated with times of day, the method further comprising setting the policy based at least in part on a time of day of when the mood is determined.

6. The method of claim 4, wherein the policy includes seasonal configuration preferences, the method further comprising setting the policy based at least in part on a current season of when the mood is determined.

7. The method of claim 4, wherein the policy includes holiday configuration preferences, the method further comprising setting the policy based at least in part on a current holiday period of when the mood is determined.

8. The method of claim 1, further comprising:
comparing said monitored physiological conditions to psychological profiles;
determining an applicable psychological profile based at least in part on said comparing; and
performing said determining the mood based at least in part on the applicable psychological profile.

9. The method of claim 8, further comprising maintaining a history of monitored physiological conditions of the user.

10. The method of claim 9, further comprising determining the applicable psychological profile based at least in part on the history.

11. The method of claim 1, further comprising:
receiving user feedback concerning the configuration, the feedback at least indicating a level of approval for the configuration of the configurable machine; and
recording a history of approval for at least selected configurations for the configurable machine.

12. The method of claim 11, wherein said changing a configuration of the configurable machine is based at least in part on the history of approval.

13. The method of claim 1, further comprising:
providing monitored physiological conditions of the user to a remote manager;
receiving a remote configuration from the remote manager; and
changing the configuration based at least in part on the remote configuration.

14. The method of claim 1, further comprising providing said monitored physiological conditions to a remote manager configure to perform said determining the mood for the user.

15. The method of claim 1, further comprising monitoring selected ones of: a gait of the user, physical movement of the user, and eating patterns of the user, and determining the mood based at least in part on said selected ones of monitoring.

16. A system providing a mood sensing environment containing adjustable machines dynamically responsive to user mood changes, comprising:
a sensor associated configured to monitor one or more physiological conditions of a user;
a first machine including a database storing data for use in correlating moods with monitored physiological conditions of the user;
a mood determinator communicatively coupled with the first machine for determining a mood for the user based at least in part on said monitored physiological conditions;
a configurable machine having a configuration determined based at least in part on the mood determined for the user.

17. The system of claim 16, wherein monitored physiological conditions of the user are provided to a remote manager configured to determine the configuration based on said physiological conditions.

18. An article comprising a machine-accessible tangible medium having one or more associated instructions for a mood sensing environment containing adjustable machines to dynamically respond to mood changes of a user, wherein the one or more instructions, if executed, results in a machine performing
associating one or more sensors with a user, at least one of said sensors monitoring one or more physiological conditions of the user;
determining a mood for the user based at least in part on monitored physiological conditions of the user;
selecting a configurable machine of the environment; and
changing a configuration of the configurable machine based at least in part on the determined mood of the user.

19. The article of claim 18 wherein the machine-accessible media further includes instructions, when executed, results in the machine performing automatically changing the configuration responsive to changes in the user's mood.

20. The article of claim 18 wherein the machine-accessible media further includes instructions, when executed, results in the machine associating a body area network with the user, the body area network including selected ones of said one or more sensors associated with the user.

21. The article of claim 18 wherein the machine-accessible media further includes instructions, when executed, results in the machine performing:
determining a policy applicable to the mood sensing environment; and
changing the configuration in accord with the policy.

22. The article of claim 18 wherein the machine-accessible media further includes instructions, when executed, results in the machine performing:
comparing said monitored physiological conditions to psychological profiles;
determining an applicable psychological profile based at least in part on said comparing; and
performing said determining the mood based at least in part on the applicable psychological profile.

23. The article of claim 18 wherein the machine-accessible media further includes instructions, when executed, results in the machine performing:
maintaining a history of monitored physiological conditions of the user; and
determining the applicable psychological profile based at least in part on the history.

24. The article of claim 18 wherein the machine-accessible media further includes instructions, when executed, results in the machine performing:
receiving user feedback concerning the configuration, the feedback at least indicating a level of approval for the configuration of the configurable machine; and
recording a history of approval for at least selected configurations for the configurable machine.

25. The article of claim 18 wherein the machine-accessible media further includes instructions, when executed, results in the machine performing:
providing monitored physiological conditions of the user to a remote manager;
receiving a remote configuration from the remote manager; and
changing the configuration based at least in part on the remote configuration.
